(19) United States
(12) Patent Application Publication
(10) Pub. No.: US 2006/0030861 A1
(43) Pub. Date: Feb. 9, 2006

(54) Methods and devices for retracting tissue in minimally invasive surgery

(76) Inventors: Robert E. Simonson, Boca Raton, FL (US); Eric Kolb, Milton, MA (US); David Konieczynski, Needham, MA (US); Jonathan Fanger, Fall River, MA (US); Michael Zajack, Marshfield, MA (US); Michael Jacene, Blackstone, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/185,921

(22) Filed: Jul. 20, 2005

Related U.S. Application Data
(60) Provisional application No. 60/589,727, filed on Jul. 21, 2004.

(51) Int. Cl. A61F 2/34

(52) U.S. Cl. 606/99

(57) ABSTRACT

Minimally invasive methods and devices are described for providing access to a surgical site proximate the anterior region of a patient's spine. In an exemplary embodiment, the device is a cannula that includes a distal end adapted to mate with the anterior surface of a vertebra. An exemplary method includes positioning the cannula through an incision, placing the distal end of the cannula against the anterior surface of a vertebra, and performing a surgical procedure through the cannula. Instruments or spinal implants may be inserted through the cannula.

### CROSS-REFERENCE TO RELATED APPLICATION

This application claims priority to U.S. Provisional Application No. 60/589,727, filed Jul. 21, 2004, incorporated herein by reference.

### BACKGROUND

This application relates to instruments for use in spinal surgery, and in particular to minimally invasive methods and devices for accessing and introducing spinal implants and instruments to a location proximate the spine.

In anterior spine surgery, surgeons typically employ blunt dissection of the tissues surrounding the cervical spine to provide initial access to the cervical spinal anatomy. After dissection, the tissue is typically expanded to facilitate access to the cervical vertebrae and disks. Conventional methods and instruments for expansion of the tissue proximate the cervical spine may cause significant trauma to the expanded tissue. For example, retractor blades may be placed under the longus colli muscles that run bilaterally along the anterior cervical spine. The retractor blades can be expanded with, for example, a mechanical ratcheting retractor frame. The retractor blades are often opened without any opportunity to measure the amount of retraction force being placed on the esophageal tissue, which can result in damage to the esophageal tissue due to excessive retraction force.

In other techniques, a surgical assistant may manually hold the retractor blades open as the surgeon performs the procedure. During surgery it is common for one of the blades to slip out from under the muscles, allowing tissue, such as muscle, to creep into the surgeon's visual field and requiring the surgeon to reposition the blades to capture the creeping tissue. When this occurs, the retracted tissue may be exposed to differing amounts of retraction force, which can result in increased trauma to the retracted tissue.

Recently, the trend in spinal surgery has been moving toward providing devices for minimally invasive access and methods for implanting spinal devices. For example, U.S. Pat. No. 6,159,179, US Patent Application Publication Number 2003-0083688,

and US Patent Application Publication Number 2003-0083689, which are hereby incorporated by reference, disclose systems of dilators and retractors to provide minimally invasive access to the spine. While such systems may be used in any area of the spine and offer advantages over the prior art invasive retractors that required open incisions to access the surgical site, such systems may not be optimal for use in the anterior spine. Accordingly, there remains a need for improved minimally invasive access devices and methods for introducing surgical instruments and/or spinal implants to the anterior spinal anatomy.

### SUMMARY

Disclosed herein are devices for providing minimally invasive access to the anterior spine and methods for positioning instruments and spinal implants proximate to the anterior spine.

In one exemplary embodiment, a method for accessing a surgical site on a patient's anterior spinal column may comprise making an incision in the patient, expanding the incision to create a pathway from the incision to a surgical site proximate an anterior surface of a first vertebra and an anterior surface of a second vertebra, and advancing a cannula through the pathway to the surgical site. The cannula, in the exemplary embodiment, may have a proximal end positioned outside the patient's body, a distal end adapted to correspond to a curvature of the anterior surface of the first vertebra and the anterior surface of the second vertebra, and a channel extending between the proximal and distal ends of the cannula. The exemplary method may further include positioning the distal end of the cannula against the anterior surface of the first vertebra and the anterior surface of the second vertebra.

In another exemplary embodiment, a cannula may comprise a proximal end, a distal end configured to correspond to the curvature of an anterior surface of a vertebra, and a sidewall defining a channel between the proximal end and the distal end and defining a longitudinal axis.

In another exemplary embodiment, a system for minimally invasive spine surgery may comprise a first dilator having a first diameter and a cannula. The cannula, in the exemplary embodiment, may comprise a proximal end, a distal end spaced apart a distance from the proximal end, and a sidewall defining a channel extending from the proximal end to the distal end. In the exemplary system, the distal end may be configured to correspond to the curvature of an anterior surface of a vertebra and the lumen may have a length sufficient to at least span from a skin incision to proximate a vertebra and a diameter greater than the first diameter.

In another exemplary embodiment, a cannula may comprise a proximal end, a distal end, and a sidewall defining a channel between the proximal end and the distal end. In the exemplary embodiment, the sidewall may include a distal edge that defines the distal end of the cannula. The distal edge, in the exemplary embodiment, may have a first segment having a curvature that approximates a curvature of an anterior surface of a vertebra.

### BRIEF DESCRIPTION OF THE DRAWINGS

These and other features and advantages of the methods and instruments disclosed herein will be more fully understood by reference to the following detailed description in conjunction with the attached drawings in which like reference numerals refer to like elements through the different views. The drawings illustrate principles of the methods and instruments disclosed herein and, although not to scale, show relative dimensions.

FIG. 1 is a front view of an exemplary cannula;

FIG. 2 is a side view of the cannula illustrated in FIG. 1;

FIG. 3 is a top view of the cannula of FIG. 1;

FIG. 4 is a front view of an exemplary cannula;

FIG. 5 is a side view of the cannula illustrated in FIG. 4;

FIG. 6 is a top view of the cannula of FIG. 4;

FIG. 7A is a sagittal view of an exemplary cannula being positioned against the anterior surface of a first and second cervical vertebra and spanning the disc space between the vertebrae;

FIG. 7B is a view of the cannula illustrated in FIG. 7A taken in the transverse plane;

FIG. 8 is a front view of an exemplary embodiment of a cannula having diametrically opposed cutout portions;

FIG. 9 is a side view of the cannula of FIG. 8;

FIG. 10 is a top view of the cannula of FIG. 8;

FIG. 11 is a sagittal section view of an exemplary cannula positioned against the anterior surface of a first and second cervical vertebra, illustrating a spinal implant, such as a cervical plate, being inserted through a cannula;

FIG. 12 is a front view of the distal end of another exemplary embodiment of a cannula;

FIG. 13 is a front view of the distal end of another exemplary embodiment of a cannula; and

FIG. 14 is a side view of the distal end of another exemplary embodiment of a cannula;

FIG. 15A is a bottom view of the distal end of another exemplary embodiment of a cannula, illustrating slotted tabs for engaging distraction and/or alignment pins, such as Caspar pins;

FIG. 15B is a top view of another exemplary embodiment of a cannula, illustrating distraction pins and a cervical plate positioned within the cannula;

FIG. 15C is a top view of another exemplary embodiment of a cannula including slots for receiving distraction pins;

FIG. 16A is a side view of another exemplary embodiment of a cannula, illustrating an proximal opening in the cannula for facilitating positioning of instruments through the cannula;

FIG. 16B is a side view of another exemplary embodiment of a cannula, illustrating a proximal end of the cannula that is rotatable relative to the distal end of the cannula to facilitate positioning of a proximal opening in the cannula;

FIG. 17A is a side view of another exemplary embodiment of a cannula, illustrating a proximal end of the cannula tapering from a proximal increased extent to a reduced extent;

FIG. 17B is a side view of another exemplary embodiment of a cannula, illustrating a portion of the proximal end of the cannula tapering from a proximal increased extent to reduced extent;

FIGS. 18A and 18B are top views of alternative embodiments of a cannula having one or more drill guides integral to the cannula;

FIG. 19 is a side view of another exemplary embodiment of a cannula, illustrating a proximal slot provided in the cannula to facilitate connection of an instrument to the cannula;

FIGS. 20 and 21 are side views of exemplary embodiment of cannulas including anchors for anchoring the cannula to one or more vertebra;

FIG. 22 is a front view of another exemplary embodiment of a cannula including a feet for facilitating retraction of tissue during positioning of cannula;

FIG. 23 is a top view of the cannula of FIG. 22;

FIG. 24 is a front view of another exemplary embodiment of a cannula including an asymmetrical proximal segment; and

FIG. 25 is a front view of another exemplary embodiment of a cannula having a reduced sized proximal segment.

### DETAILED DESCRIPTION OF EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Disclosed herein are minimally invasive methods and devices for accessing the anterior spinal column and introducing instruments and/or spinal implants to a surgical site proximate the anterior spinal column. In general, an exemplary method involves inserting a cannula contoured at the distal end to approximate the curvature of the anterior surface of a vertebra to create a pathway that extends from an incision, such as, for example, a minimally invasive incision, to a surgical site proximate the anterior spine. In an exemplary embodiment, a cannula is used to create a minimally invasive pathway for receiving spinal instruments and for delivering one or more spinal implants, or components thereof, to a surgical site on the anterior spine. A spinal implant, such as a bone anchor, a fixation element, e.g., a rod, plate, or tether, a graft containment device, such as, for example, a strap or buttress staple, and/or an interbody fusion device, may be inserted through the cannula, depending on the size and shape of the cannula, in any orientation, including for example, parallel or perpendicular to the spine. The spinal implant may then be oriented and positioned to couple the implant to the spine. A fastening element or other coupling mechanism, if necessary, may be introduced through the cannula to mate the spinal implant to the spine.

The methods and devices disclosed herein are particularly suited for use with a minimally invasive percutaneous incision for accessing the anterior region of the spinal column. Minimally invasive incisions minimize damage to intervening tissues, and can, thus, reduce recovery time and post-operative pain. The methods and devices disclosed herein permit the delivery of one or more spinal implants along a minimally invasive pathway, thus eliminating the need to create a large working area at the surgical site.

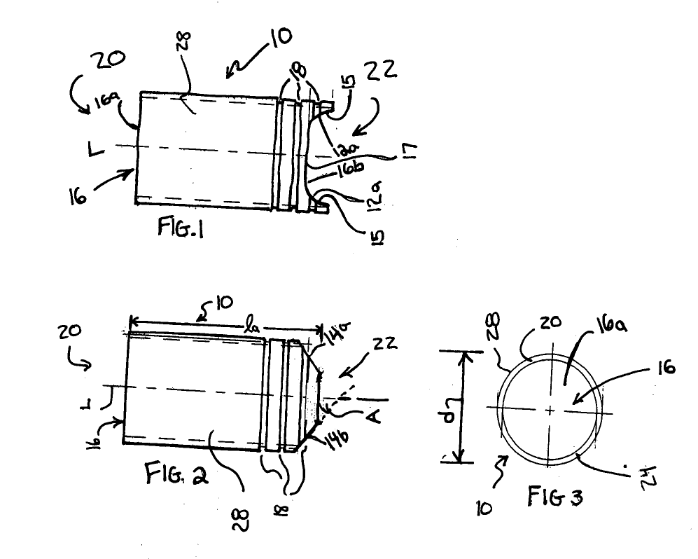

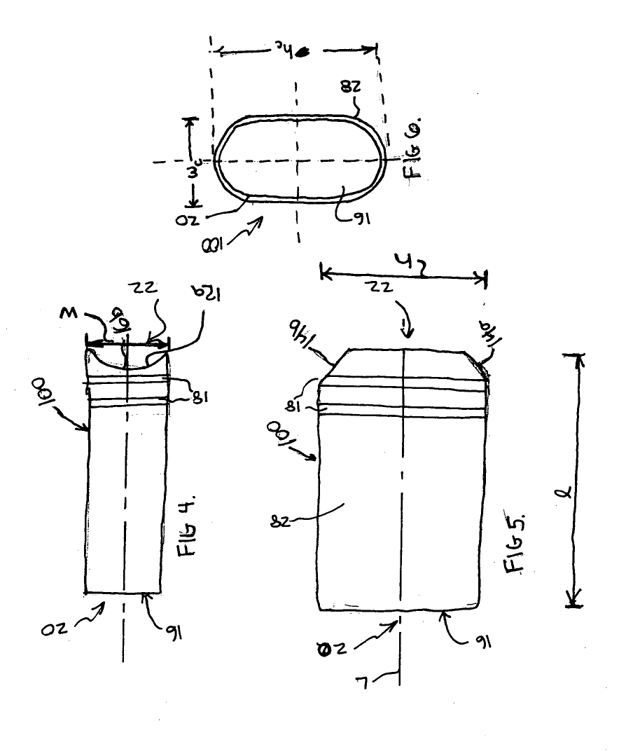

FIGS. 1-3 illustrate an exemplary embodiment of a cannula 10 for providing minimally invasive access to the spine, in particular, to the anterior spine. As used herein, the term “anterior” and “anterior spine” generally refers to an approach to the spine through the front or side of the patient, e.g., to the front of or along the coronal plane, typically while the patient is in a supine position or a lateral position, and the anatomy of the spine that is accessible through such an approach. The exemplary cannula 10 includes a proximal end 20, a distal end 22, and a sidewall 24 defining a channel 16 and a longitudinal axis L, each extending between the proximal end 20 and the distal end 22. In use, the channel 16 of the exemplary cannula 10 provides unobstructed access from the proximal end 16a of the channel 16 to the distal end 16b of the channel 16 to permit the advancement of instruments and/or implants through the cannula 10.

The size of the exemplary cannula 10 may vary depending on the intended use of the cannula 10, for example, the region of the spine, e.g., cervical, thoracic, or lumbar, and the type(s) of implants and instruments desired to be positioned through the channel 16 of the cannula 10. In certain exemplary embodiments, for example, the cannula 10 may have a length lₐ sufficient to span from a skin incision to proximate a vertebra. The length lₐ of the cannula 10 may be varied, for example, depending of whether the cannula 10 is designed for use in the cervical, thoracic, or lumbar spine. For example, the cannula 10 may have a length lₐ that allows the proximal end 16a of the cannula 10 to be positioned outside the patient's body, e.g., proximal to or parallel to the level of the skin, while the distal end 16b of the cannula 10 is in proximity to or abuts against the anterior surface of a vertebra. For the cervical spine, for example, the length lₐ of the cannula may be between approximately 15 mm and approximately 100 mm, and preferably is between approximately 20 mm and approximately 60 mm. For the thoracic spine, for example, the length lₐ of the cannula may be between approximately 50 mm and approximately 350 mm, and preferably is between approximately 200 mm and approximately 300 mm. For the lumbar spine, for example, the length lₐ of the cannula may be between approximately 100 mm and approximately 400 mm, and preferably is between approximately 125 mm and approximately 200 mm. The cannula 10 may include indicia on the outer surface 28 indicating the length of from the distal end 22 of the cannula 10.

In certain exemplary embodiments, the cannula 10 may be configured to provide a minimally invasive pathway for the delivery of one or more instruments and/or implants to the spine. For example, the cannula 10 may be sized and shaped for implantation through a minimally invasive incision, which is a relatively small incision that typically has length that approximates the diameter or width of the device being inserted therethrough.

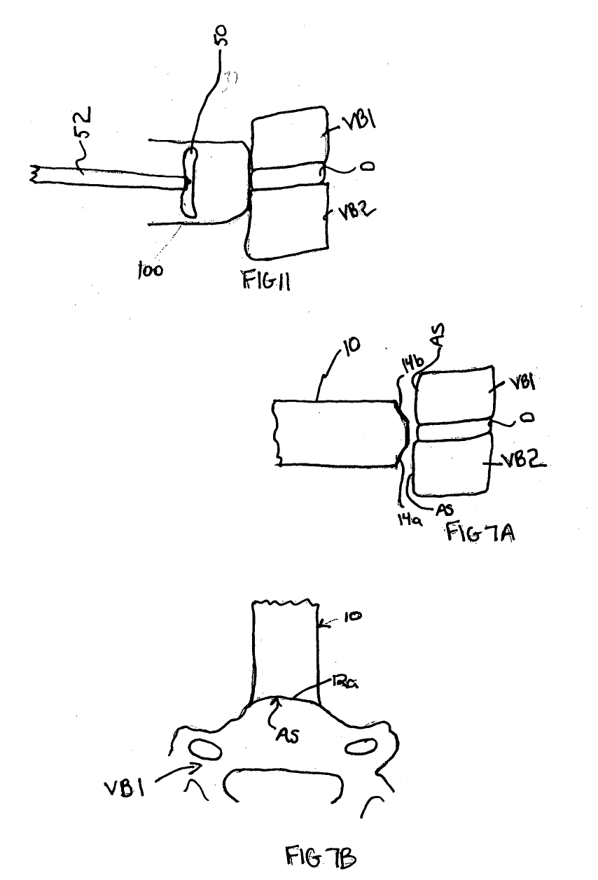

Continuing to refer to FIGS. 1-3, the exemplary cannula 10 may have a cross-sectional shape and size that varies on depending on the intended use of the cannula 10, for example, the region of the spine, e.g., cervical, thoracic, or lumbar, and the type(s) of implants and instruments desired to be positioned through the channel 16 of the cannula 10. In the exemplary embodiment illustrated in FIGS. 1-3, for example, the exemplary cannula 10 has a circular cross section. In other exemplary embodiments, such as the exemplary cannula illustrated in FIGS. 4-6, the cannula may have an elliptical or oval cross-section. One skilled in the art will appreciate that the cannula may have be have a cross section that is circular, rectangular, square, elliptical, polygonal or any other shape suitable for creating a pathway from the skin to proximate the spine. In the exemplary embodiments illustrated in FIGS. 1-3 and 4-6, the cannula has a generally constant cross section, e.g., the size and/or shape of the cross section of the cannula does not vary along the length lₐ of the cannula. In certain other exemplary embodiments, the cross section of the cannula may vary in size and shape along the length lₐ of the cannula. For example, the width or diameter of the cannula may vary along the length of the cannula. In the exemplary embodiments illustrated in FIGS. 1-3 and 4-6 the cannula has a continuous cross section. In certain other embodiments, the cannula may have a non-continuous cross-section. For example, the cannula may have a C-shaped cross section or may include one or more longitudinally oriented slots that interrupt the cross section along the length of the cannula.

In the embodiment illustrated in FIGS. 1-3, the channel 16 of the exemplary cannula 10 may have a diameter d that is sufficient to allow a spinal implant and/or instrument to be introduced therethrough. Examples of spinal implants that may be introduced through cannula 10 include spinal fixation elements, such as a plate, rod, or tether, interbody fusion devices, nucleus replacement devices, artificial discs, and fasteners, such as bone anchors. The diameter d of the channel 16 may be sized to allow any of these implants and associated instruments to be introduced therethrough. For example, the diameter d of the channel 16 of the exemplary cannula 10 may be between approximately 5 and approximately 50 mm, and preferably is between approximately 7 mm and approximately 25 for the cervical spine and implants designed for use in the cervical spine. For example, the diameter d of the channel 16 of the exemplary cannula 10 may be between approximately 10 mm and approximately 50 mm, and preferably is between approximately 12 mm and approximately 45 for the thoracic spine and implants designed for use in the thoracic spine. For example, the diameter d of the channel 16 of the exemplary cannula 10 may be between approximately 20 mm and approximately 60 mm, and preferably is between approximately 30 mm and approximately 45 for the lumbar spine and implants designed for use in the lumbar spine.

In certain exemplary embodiments, the diameter d of the exemplary cannula 10 may be sized to span between a first vertebra and a second vertebra to provide access to the first vertebra, the second vertebra and the disk therebetween.

The exemplary cannula 10 may be constructed from any material suitable for use in vivo, including metals, such as stainless steel, aluminum, or titanium, polymers, ceramics, or composite materials. In certain exemplary embodiments, the cannula 10 may be constructed from a translucent polymer.

The outer surface 28 of the exemplary cannula 10 may be contoured to prevent any sharp edges and to minimize injury to muscles and tissues surrounding the cannula 10. In addition, the outer surface 28 of the cannula 10 may include surface texturing to facilitate holding retracted tissue in place, in particular, away from the distal end 16b of the channel 16. The surface texturing may be, for example, one or more annular grooves 18 formed in the outer surface 28 of the cannula 10. In certain embodiments, the surface texturing may be surface roughening, ridges, spiral grooves, and/or materials with a high coefficient of friction. In certain exemplary embodiments, the outer surface 28 of the cannula is coated with silicon to facilitate holding retracted tissue. For example, a sheath of silicon or other material with a high coefficient of friction may be positioned about the distal end 22 of the cannula 20. In other exemplary embodiments, a ring of silicon or other material with a high coefficient of friction may be positioned within one or more of the grooves 18. Alternatively, the cannula may include a deformable feature, such as a barb, that deflects upon insertion of the cannula and exerts a spring force on the tissue to retain the cannula in position. In the case of a cannula constructed from a polymer material, a ring of radio-opaque material, such as a metal ring, may be positioned in one or more of the grooves 18 to permit radiographic visualization of the cannula 10.

In the illustrated embodiment, the exemplary cannula 10 has a distal end 22 that may be configured to correspond to the size and shape of an anterior surface of a vertebra to facilitate tissue retraction at the distal end 22 of the cannula 10 and inhibit tissue creep, i.e. movement of retracted tissue distal to the distal end 22 of the cannula 10 that may occlude the distal end 16b of the channel 16. In certain exemplary embodiments, for example, the distal end 22 of the exemplary cannula 10 may be configured to correspond to the curvature of an anterior surface of a vertebra, for example, the anterior surface of a vertebral body of a vertebra. At least a portion of the distal end 22 may have a curvature approximate to the curvature of an anterior surface of a vertebra in sagittal plane and/or at least a portion of the distal end 22 may have a curvature approximate to the curvature of an anterior surface of a vertebra in the transverse plane.

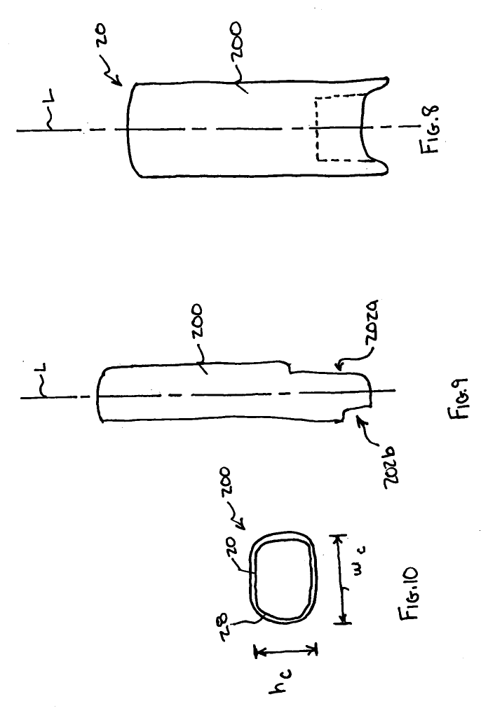

For example, the distal end 22 may have a first segment 12a that has a shape that approximates the curvature of the anterior surface of a vertebra. Referring to the FIGS. 1 and 7b, which illustrates the exemplary cannula 10 adjacent a first vertebra VB1, the first segment 12a may have a curvature that approximates the transverse curvature of the anterior surface AS of the vertebral body of the first vertebrae VB1. The first segment 12a may be arcuate in shape and may have a constant radius or, in other exemplary embodiments, may comprise a plurality of arcuate sections, with differing radii, a plurality of linear sections oriented at differing angles with respect to each other, or a combination of arcuate and linear sections. In the exemplary embodiment illustrated in FIG. 1, the first segment 12a comprises two arcuate sections 15 separated by a linear section 17.

The distal end 22 may also have a second segment positioned, for example, diametrically opposite the first segment, and having a shape corresponding to the curvature of an anterior surface of a vertebra. For example, the second segment may have a curvature approximate to the transverse curvature of the anterior surface AS of the vertebral body of the first vertebrae VB1 or the anterior surface of a second vertebra. In certain exemplary embodiments, the first segment 12a and the second segment 12b may be analogously shaped such that, for example, the first segment 12a and the second segment 12b may have a common curvature. For the cervical spine, for example, the radius of the first segment 12a may be approximately 5 to approximately 30 mm, and preferably is between approximately 15 mm to approximately 25 mm. For the thoracic spine, for example, the radius of the first segment 12a may be approximately 5 mm to approximately 65 mm. For the lumbar spine, for example, the radius of the first segment 12a may be approximately 10 mm to approximately 65 mm and preferably is between approximately 20 mm and approximately 40 mm.

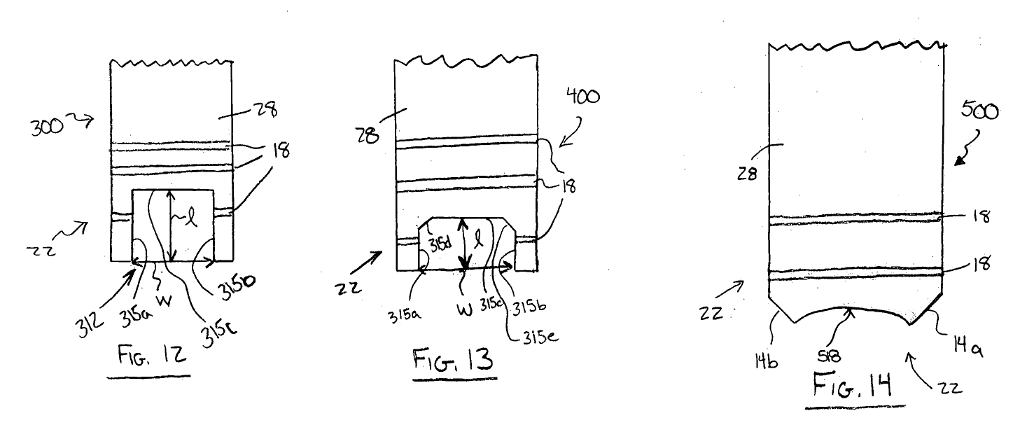

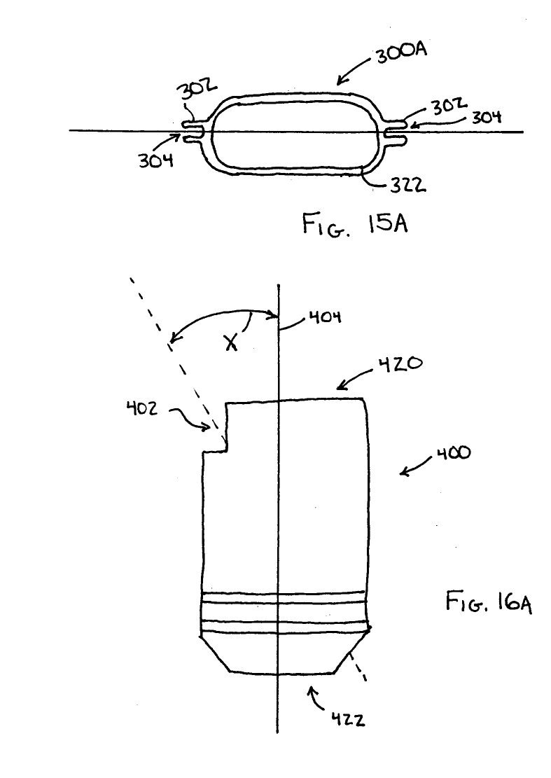

FIGS. 12 and 13 illustrate exemplary alternate shapes for the distal end of the cannula. FIG. 12 illustrates a cannula 300 having a distal end 22 including a segment 312 having a shape that approximates the transverse curvature of an anterior surface of a vertebra. The segment 312, in the exemplary cannula 300, comprises three linear sections, namely a first section 315a and second section 315b, each oriented approximately parallel to the longitudinal axis L of the cannula 300, and a third section 315c interposed between the first section 315a and second section 315b and oriented generally perpendicular to the first section 315a and second section 315b. The length l of the first and second sections 315a, 315b and the width w of the third section 315c may be selected to correspond to the curvature of an anterior surface of a vertebra. In certain exemplary embodiments, for example, length l of the first and second sections 315a, 315b and the width w of the third section 315c may be selected such that each section contacts a portion of an anterior surface of a vertebra when the distal end 22 is brought into contact with the vertebra.

FIG. 13 illustrates a cannula 400 having a distal end 22 including a segment 412 having a shape that approximates the transverse curvature of an anterior surface of a vertebra. The segment 412, in the exemplary cannula 400, comprises five linear sections, namely a first section 415a and second section 415b, each oriented approximately parallel to the longitudinal axis L of the cannula 400, a third section 415c oriented generally perpendicular to the first section 415a and second section 415b, and a fourth section 415d and fifth section 415e, oriented at an angle to the first section 415a and second section 415b. The length l of the first and second sections 415a, 415b, the width w of the third section 415c, and the angle of the fourth and fifth sections 415d,e may be selected to correspond to the curvature of an anterior surface of a vertebra. In certain exemplary embodiments, for example, length l of the first and second sections 415a, 415b, the width w of the third section 415c, and the angle of the fourth and fifth sections 415d,e may be selected such that each section contacts a portion of an anterior surface of a vertebra when the distal end 22 is brought into contact with the vertebra.

Continuing to refer to FIGS. 1-3, the cannula 10 may have a third segment 14a having a curvature that approximates the curvature of an anterior surface of a vertebra. Referring to FIGS. 2 and 7A, which illustrates an exemplary cannula being positioned against a first vertebra VB1 and a second vertebra, the third segment 14a may have a curvature that approximates the sagittal curvature of the anterior surface AS of at least a portion of the vertebral body of the first vertebrae VB1. The third segment 14a may be arcuate in shape and may have a constant radius or, in other exemplary embodiments, may comprise a plurality of arcuate sections, with differing radii, one or more linear sections oriented at differing angles with respect to each other, or a combination of arcuate and linear sections. In the illustrated exemplary embodiment, the third segment 14a comprises one linear section oriented at an angle A to the longitudinal axis L of the cannula 10. The cannula 10 may have a fourth 14b segment having a curvature that approximates the sagittal curvature of the anterior surface AS of at least a portion of the vertebral body of the second vertebrae VB2. In certain exemplary embodiments, the third segment 14a and the fourth segment 14b may be analogously shaped such that, for example, the third segment 14a and the fourth segment 14b may oriented at a common angle to the longitudinal axis L of the cannula 10.

FIG. 14 illustrates an exemplary cannula 500 having a distal end 22 including a segment 518 having a shape that approximates the sagittal curvature of an anterior surface of one or more vertebrae. In the cervical spine, for example, the segment 518 may be arcuate in shape and have a curvature generally corresponding to lordosed anterior surface of one or more vertebrae. The segment 518 may have a radius of approximately 100 mm to approximately 300 mm and preferably has a radius of approximately 180 mm for the cervical spine.

Referring to FIGS. 4-6, an exemplary embodiment of a cannula 100 having elliptical or oval cross section. The exemplary elliptical cannula 100 may be analogous in construction to the exemplary circular cannula 10 described above, except for the shape of the cross-section. The exemplary cannula 100 may have a width w_{c} and a height h_{c} that varies depending on the intended use of the cannula 100, for example, the region of the spine, e.g., cervical, thoracic, or lumbar, and the type(s) of implants and instruments desired to be positioned through the channel 16 of the cannula 100. For example, the cannula 100 may have a width w_{c} and a height h_{c} that are sufficient to allow a spinal instrument and/or a spinal implant to be introduced therethrough. For the cervical spine, for example, the cannula 100 may have a width w_{c} between approximately 5 mm and approximately 50 mm, preferably between 7 mm and 25 mm. For the thoracic spine, for example, the cannula 100 may have a width w_{c} between approximately 10 mm and approximately 50 mm, preferably between 12 mm and 45 mm. For the lumbar spine, for example, the cannula 100 may have a width w_{c} between approximately 20 mm and approximately 60 mm, preferably between 30 mm and 45 mm.

The height h_{c} of the exemplary elliptical cannula 100 may be approximately equal to or greater than the width w_{c} of the cannula 100. In the illustrated embodiment, the exemplary cannula 100 has a height h_{c} that is sufficient to span the disc space between two adjacent vertebrae and abut against the anterior surface of each vertebral body. Such as configuration is particularly suited for the positioning of a plate relative to the two adjacent vertebrae. FIG. 11 illustrates the exemplary cannula 11 positioned against two adjacent vertebrae, vertebra VB1 and vertebra VB2 and the delivery of a plate 50 through the cannula 100 with a plate insertion instrument 52. The exemplary cannula 100 permits removal of the disk D between the vertebrae, insertion of an interbody fusion device, and the placement of a fixation element, e.g., plate 50, through the channel 16 of the cannula 100. In such applications, the height h_{c} of the cannula may be equal to or greater than the height of the plate to allow the plate 50 to be delivered in an orientation that is generally transverse to the longitudinal axis L of the cannula L. The height h_{c} of the cannula may be varied depending on the number of levels and the region of the spine. For example, in the cervical spine, the height h_{c} of the cannula may be approximately 5 mm to approximately 75 mm and is preferably approximately 7 mm to approximately 50 mm. In the case of a one level fusion of two adjacent cervical vertebrae, the height h_{c} of the cannula may be approximately 25 mm to approximately 30 mm, to, for example, accommodate a cervical plate of 20 mm to 30 mm in length. In other exemplary embodiments, the height h_{c} of the cannula may also be sufficient to span three or more adjacent vertebrae. In the thoracic and lumbar spine, the height h_{c} of the cannula may be approximately 10 mm to approximately 200 mm, depending on the number of levels being treated.

FIGS. 8-10 illustrate another exemplary embodiment of a cannula 200 having a generally elliptical cross section. The exemplary cannula 200 may be analogous in size and shape to the cannula 100 described above. The cannula 200 may include one or more cut-out portions 202 formed in the sidewall 24 of the cannula 200. The cut out portion 202 provide a passageway from the channel 16 for the subcutaneous positioning of instruments and implants. In the illustrated embodiment, the exemplary cannula 200 includes a first cut out portion 202a and a second cut out portion 202b positioned opposite the first cut out portion 202a. Any number of cut out portions may be provided in the cannula. The cut out portions 202a, 202b may have the same length along the longitudinal axis L of the cannula or, as in the illustrated embodiment, the cut out portions may have different lengths. In certain exemplary embodiments, the cut-out portions permit the subcutaneous positioning of a spinal fixation element, such as a plate, between adjacent vertebrae.

In certain exemplary embodiments, a plurality of cannulas, such as one or more of the cannulas described above, may be provided in a minimally invasive surgical system. In an exemplary system, cannulas of varying lengths, widths, and heights may be provided to facilitate use of the system in varying regions of the spine and with varying instruments. For example, a system may include one or more cannulas configured for the cervical spine, one or more cannulas configured for the lumbar spine, and one or more cannulas configured for the thoracic spine. In another exemplary system, one or more cannulas may be configured for a microdiscectomy procedure, one or more cannulas may be configured for delivery of an interbody fusion device, and one or more cannulas may configured for delivery of a fixation element, such as, for example, a plate.

In certain exemplary embodiments, a cannula may comprise one or more telescoping sections that allow lengthwise adjustment of the cannula.

An exemplary embodiment of a minimally invasive surgical method provides for the placement of a cannula for access to the anterior spine for preparation of the surgical site and/or implantation of a spinal implant. In the exemplary method, initially an incision may be made in the patient for placement of the cannula. The incision may be a minimally invasive incision made in the patient's skin that is expanded, for example, by retraction and/or dilation, to create a pathway from the incision to a surgical site proximate an anterior surface of a first vertebra. The location, size, shape, amount and orientation of expansion of the incision may depend on the procedure being performed and the type of implants being inserted. The instruments and spinal implants employed during the procedure may be advanced through the cannula to the surgical site proximate to an anterior surface of the first vertebra VB1.

The incision may be expanded to create the pathway in any conventional manner. In certain embodiments, for example, the incision may be expanded by dilation to the desired size, shape, and orientation. For example, the incision may be sequentially dilated using a plurality of dilators to create the pathway. Alternatively, a retractor may be inserted into the dilated incision 20 to further expand the incision and/or to define the pathway 26.

In certain exemplary embodiments, the initial incision may be expanded by inserting one or more retractors into the incision and expanding the incision to the desired size, shape, and orientation by expanding the retractor accordingly. The expanded retractor can define the pathway from the incision to proximate an anterior surface of the vertebra. Any type of conventional retractor or retractors may be employed to expand the incision. For example, suitable retractors are described in commonly owned U.S. Patent Application Publication Number 2005-0137461; U.S. Provisional Patent Application Ser. No. 60/530,655, filed Dec. 18, 2003, entitled Surgical Retractor Systems, Illuminated Cannula and Methods of Use; U.S. patent application Ser. No. 11/016,347, filed Dec. 17, 2004, entitled Surgical Retractor Systems, Illuminated Cannula and Methods of Use; U.S. patent application Ser. No. 11/016,549, filed Dec. 17, 2004, entitled Surgical Retractor Systems, Illuminated Cannula and Methods of Use; and U.S. patent application Ser. No. 10/808,687, entitled Surgical Retractor Positioning Device, each of which are incorporated herein by reference.

In certain exemplary embodiments, the surgeon may expand the incision to create the passageway using one or more fingers. In such embodiments, a cannula may be positioned on the surgeon's finger during dilation and advanced into position after dilation using the finger as a guide.

An alternate method may include percutaneously positioning a cannula through a skin incision. The incision is preferably a percutaneous skin incision that has a shape and extent that is typically equal to, or slightly greater than, the extent of the instruments and implants being inserted thereto. In certain exemplary embodiments, for example, the incision may be a stab incision that is expanded to facilitate positioning of the cannula therethrough.

The cannula may be advanced through the incision and the pathway to the surgical site proximate a vertebra. The distal end of the cannula may be positioned against an anterior surface of the vertebra, for example, an anterior surface of the vertebral body of the vertebra. Preferably, the cannula has a distal end configured to correspond to the curvature of the anterior surface of the vertebra, such as, for example, the exemplary cannulas described above. An instrument may be used to move tissue away from the distal end of the cannula during positioning of the cannula. The instrument, such as a tissue retractor or the like, may be positioned within the cannula and/or may be positioned external to the cannula to move tissue away from the distal end of the cannula. Once the cannula is positioned, one or more instruments and/or implants may be positioned through the cannula to perform a procedure at or proximate the vertebra. Exemplary procedures include removal of disk material, dissection and/or removal of a portion of the vertebra, placement of one or more implants relative to the vertebra or the adjacent disk space.

FIG. 11 illustrates a minimally invasive method of implanting a spinal implant, e.g., a plate 50, through a cannula, such as, for example, cannula 100 described above. While the method is shown and described in connection with the insertion of a plate through the cannula 100, a person skilled in the art will appreciate that the exemplary method is not limited to use with such plates, and that a variety of other spinal implants known in the art such as interbody fusion devices (including bone grafts), nucleus replacement devices, artificial disc replacement devices and fasteners can be used. The method can also be performed using only some of the method steps disclosed herein, and/or using other methods known in the art.

In the exemplary method, the proper sized cannula is selected based upon the implant to be implanted and the location on the spine where the implant is to be implanted. The selected cannula 100 may be placed in a skin incision and advanced to the anterior surfaces of the first and second vertebrae, as discussed above. Once the distal end 22 of the cannula 100 is positioned against the anterior surfaces of the vertebrae, the plate 50 may be positioned in the channel 16 of the cannula 100 and advanced to the vertebrae using a suitable instrument 52. Examples of instruments used to hold and insert the plate are described in U.S. Patent Application Publication Number 2004-0204710; U.S. Patent Application Publication Number 2004-0267274; U.S. Patent Application Publication Number 2005-0059975; and U.S. Patent Application Publication Number 2004-0204716; each of which are hereby incorporated by reference. In the exemplary method, the plate 50 is advanced in an orientation substantially perpendicular to the longitudinal axis L of the cannula 110 and in an orientation substantially parallel to the spine. Once the plate 50 is in position against the anterior surface of the vertebrae, the plate may be anchored to the vertebrae by suitable bone anchors that are advanced to the vertebrae through the channel 16 of the cannula 100.

In another exemplary method, the cannula 200 illustrated in FIGS. 8-10 may be employed to deliver an implant and/or an instrument to an anterior surface of a vertebra. The cutout portions 202a and 202b, described above, allow the advancement of an implant, such as fixation element, e.g., a plate, through the channel 16 of the cannula 200 in a reduced profile orientation, for example, in an orientation substantially parallel to the longitudinal axis L of the cannula 200. As the implant approaches the distal end 22 of the cannula 200, the orientation of the implant can be manipulated to a second orientation substantially perpendicular to the longitudinal axis L of the cannula for proper alignment with the spine. The cut out portions 202a, 202b facilitate such manipulation by allowing portions of the implant to pass therethrough. An example of a manipulator instrument is disclosed in: U.S. Patent Application Publication Number 2005-131419, and U.S. Patent Application Publication Number 2005-0131420, each of which is hereby incorporated by reference.

In certain exemplary embodiments, the distal end of the cannula may be moved from one surgical site to another surgical site by manipulating the proximal end of the cannula. For example, the distal end of the cannula may be moved from a first vertebra to a second vertebra, for example, to place a bone anchor at the first vertebra and a bone anchor at the second vertebra.

In one exemplary embodiment, a cannula may be employed to facilitate a single level spinal fusion procedure. In the exemplary method, an incision may be made transverse to the axis of the spine. The incision may be expanded by blunt dissection using a dilator, a retractor, or with the surgeon's finger(s). Tissue and muscle may be retracted to create a pathway to first and second adjacent vertebrae. In particular, muscles, blood vessels, nerves, the trachea, esophagus, and the vocal cords may be laterally retracted to create a pathway to the anterior surface of a first cervical vertebra and an anterior surface of a second vertebra. Optionally, a dilator may be placed in the incision to maintain the pathway and facilitate delivery of the cannula. The cannula may in be positioned in the incision and advanced along the pathway until the distal end of the cannula is in proximity to the anterior surface of a first cervical vertebra and an anterior surface of a second vertebra. The cannula serves to retract tissue and maintain an unobstructed path from the incision to the vertebrae to conduct the fusion procedure. The vertebrae may be distracted, optionally using Caspar pins, and the disk may be removed, as well as any anterior osteophytes on the vertebrae. The nerves may be decompressed by removing any retropulsed disk material, parts of the vertebrae, and/or portions of the posterior longitudinal ligament that me be ossified. An interbody fusion device may be placed in the disk space to facilitate fusion of the vertebrae. Exemplary interbody fusion device include allograft, autograft, and/or a cage packed with morselized bone, bone growth factor, or bone marrow concentrate. Optionally, a graft containment device, such as a plate, lateral strap, or staple, may be positioned on the vertebrae. One skilled in the art will appreciate that the exemplary method may be modified for fusion of additional levels.

In alternative methods, a cervical prosthetic disc may be implanted through the cannula in the disc space to preserve motion of the vertebra.

As previously stated, a person skilled in the art will appreciate that the method can be performed in any sequence using any of the steps. Moreover, the cannulas of the present invention can be used to perform a variety of other surgical procedures not illustrated or described herein.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

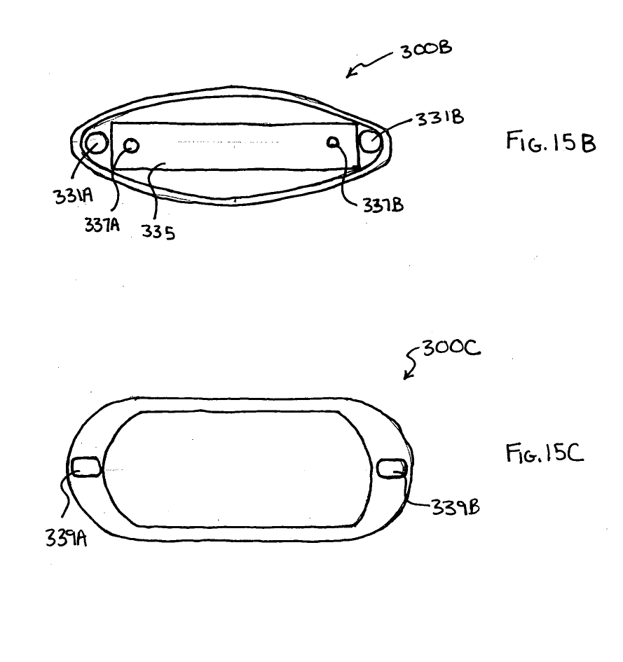

FIG. 15A illustrates another exemplary embodiment of a cannula 300A that is configured to engage alignment and/or distraction pins, such as, for example Caspar pins, positioned in the vertebra. The cannula 300A, as well as cannula 300B-C described below) may be configured in a manner analogous to one or more of the cannulas described above. In the exemplary cannula 300A, for example, the distal end 322 of the cannula 300A includes a pair of opposed tabs 302 each have a slot 304 formed therein for receiving a pin or other the like engaged in adjacent vertebra. The tabs 302, and the respective slots 304, may be aligned along a central axis 306 of the cannula 300A to facilitate alignment on the cannula 300A with the pins and the vertebra. Such a configuration may facilitate alignment of the cannula 300A along the midline of the respective vertebrae.

In certain alternative embodiments, a cannula may be configured to receive distraction/alignment pins within the cannula. Referring to FIG. 15B, for example, a cannula 300B may be elliptical, oblong, or diamond shaped to receive one or more pins, for example Caspar pins 331, within the cannula 300B. In the illustrated embodiment, the cannula 300B is sized to receive a pair of opposed Caspar pins 331A, 331B and a single level anterior cervical plate 335 having one anchor opening 337A, 337B per vertebra. In one exemplary method, the cannula 330B may be positioned in proximity to two adjacent vertebrae and a Capsar pin may be inserted through the cannula 300B into each vertebra. The vertebrae may be distracted by manipulating the Caspar pins 331A,B within the cannula 300B. After the disc is removed and a suitable interbody fusion device is positioned in the disc space, the plate 335 may be positioned against the vertebrae through the cannula 300B and the plate 335 may be anchored to each vertebra. Alternatively, the Capsar pins may be positioned in the vertebrae and the cannula 300B may be positioned over the Capsar pins, before or after distraction of the vertebrae.

In alternative embodiments, a cannula may include one or more openings within the walls of the cannula to receive distraction/alignment pins. For example, referring to FIG. 15C, the exemplary cannula 300C may include opposed openings 339A, 339B provided in the walls of the cannula 300C for receiving distraction pins such as Caspar pins. The openings 339A, 339B may extend the length of the cannula 300C. Preferably, the openings 339A, 339B have a cross section sized to permit motion of the pins within the opening to, for example, permit distraction of the vertebrae with the pins positioned in through the openings 339A, 339B of the cannula 300C. For example, the openings 339 may have a cross section that is oval, elliptical, circular, rectilinear or the like. In the illustrated embodiment, for example, the openings 339 are slotted, having an elliptical cross section.

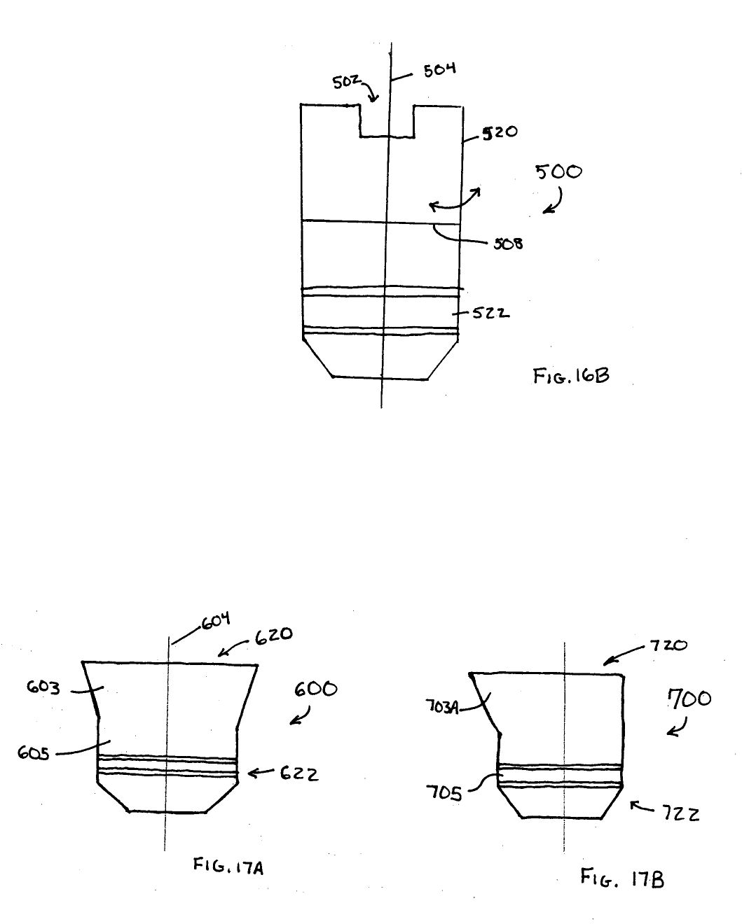

FIG. 16A illustrates another exemplary embodiment of a cannula 400 having one or more proximal windows or openings 402 that facilitate the positioning of instruments through the cannula 400. The cannula 400 may be configured in a manner analogous to one or more of the cannulas described above. One or more proximal openings 402 may be positioned at various points about the perimeter of the proximal end 420 of the cannula 400. The size and shape of each opening may be selected to permit an instrument to be positioned at an increased angle X relative to the central axis 404 of the cannula 400 to access the distal end 422 of the cannula 400 and tissue beyond the distal end 422 of the cannula 400. In the case of anterior approaches to the cervical spine, the angle X may approximately 0° to approximately 30°.

FIG. 16B illustrates another exemplary embodiment of a cannula 500 having a proximal end 520 that is rotatable about a central axis 504 of the cannula 500 and relative to the distal end 522 of the cannula 500. The cannula 500 may be configured in a manner analogous to one or more of the cannulas described above. The cannula 500 may include one or more proximal openings 502 positioned about the perimeter of the proximal end 520 of the cannula 500. Rotation of the proximal end 520 permits the position of the opening(s) 502 to be adjusted relative to the distal end 522 of the cannula 500. The intersection 508 of the proximal end 520 and the distal end 522, e.g., the length of the proximal end 520 and the distal end 522, may be varied depending on, for example, the overall length of the cannula 500 and the selected application for the cannula 500. The proximal end 520 may be connected to the distal end 522 in any manner sufficient to permit relative rotation of the proximal end 520 and the distal end 522. For example, the proximal end 520 may be connected to the distal end 522 by an interference fit or by threads.

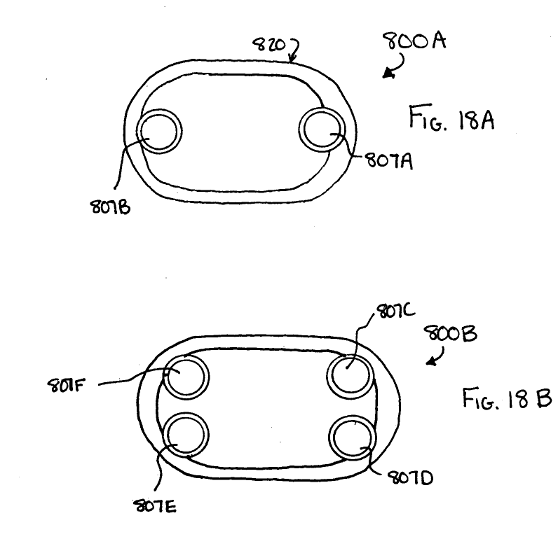

FIG. 17A illustrates an exemplary embodiment of a cannula 600 having a proximal segment 603 that tapers from an increased extent at the proximal end 620 of the cannula 600 to a reduced extent at the distal segment 605 of the cannula 600. For example, in the case of a cannula having an approximately circular cross-section, the diameter of the proximal segment 603 may taper from the proximal end 620 of the cannula to the distal segment 605. The cannula 600 may be configured in a manner analogous to one or more of the cannulas described above. The increased extent of the proximal segment 603 facilitates positioning of instruments through the cannula 600 at an increased angle to the central axis 604 of the cannula 600. In the illustrated exemplary embodiment, the distal segment 605 of the cannula 600 has a constant extent. In alternative embodiments, the distal segment 605 may taper to the distal end 622 of the cannula 600 in a manner analogous to proximal segment 603. In the illustrated exemplary embodiment, the proximal segment 603 of the cannula 600 tapers symmetrically about the central axis 604. In alternative exemplary embodiments, only a portion 703A of the proximal segment tapers from the proximal end 720 of the cannula to the distal segment 705 of the cannula 700, as illustrated in FIG. 17B.

FIG. 18A illustrates another exemplary embodiment of a cannula 800A having one or more drill guides 807 provided within the cannula 800. The cannula 800 (and 800B, illustrated in FIG. 18B) may be configured in a manner analogous to one or more of the cannulas described above. In the exemplary embodiment, the drill guides 807A and 807B are aligned along a common axis and extend from the proximal end 820 to the distal end of the cannula 800. The drill guides 807A,B may facilitate positioning and alignment of bone anchors used, for example, in connection with an anterior cervical plate. A drill, tap, anchor driver, or the like, and the bone anchor may be positioned through each drill guide. Any number of drill guides may be positioned at various locations about the perimeter of the cannula. For example, in the exemplary embodiment illustrated in FIG. 18A, the drill guide 807A is positioned to permit positioning a first bone anchor through a plate into a first vertebra and drill guide 807B is positioned to permit positioning of a second bone anchor through the plate and into a second vertebra. In alternative embodiments, additional drill guides may be provided. For example, in the cannula 800B illustrated in FIG. 18B, four drill guides 807C, 807D, 807E, 807F are provided to permit two bone anchors to be positioned per vertebra. The drill guides may be integral to the cannula or may be modular, i.e., separate components selectively connected to the cannula.

A cannula may be provided with one or more structures to facilitate connection of an instrument, such as a suction or irrigation tube, a light, or a suture. For example, an exemplary cannula 900 may include a proximal slot 915 for receiving an instrument to connect the instrument to cannula 900, as illustrated in FIG. 19. For example, the slot 915 may be sized to receive one or more sutures, which are connected at a distal end of the suture to a gauze pad or the like to absorb blood at or external to the distal end 922 of the cannula. Preferably, the slot 915 is sized to retain the suture within the slot 915. Any number of slots 915 may be provided about the perimeter of the proximal end 920 of the cannula 900. Alternatively, additional slot(s) may be sized to receive a suction tube, a light source, such as a fiber optic cable, or other instruments used with the cannula 900.

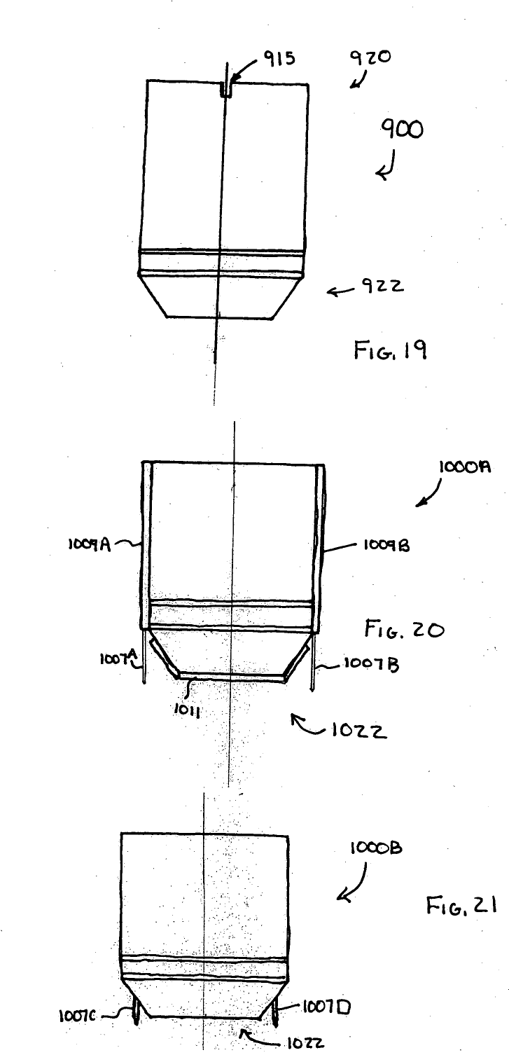

In certain exemplary embodiments, a cannula may include one or more anchors for anchoring the cannula in position, e.g., in contact with, one or more vertebra. Referring to FIG. 20, for example, a cannula 1000A may include one or more anchors 1007A, 1007B that can be positioned to project from the distal end 1022 of the cannula 1000A into one or more vertebra. The anchors 1107A, 1007B may be smooth spikes, threaded screws, staples, or any other device suitable for engaging bone. In certain exemplary embodiments, the bone anchors 1007 may be adjustable relative to the cannula 1000A to allow the anchors 1007A,B to be selectively positioned into contact with the vertebra. Referring to FIG. 20, for example, the anchors 1007A, 1007B each may be housed in a tubular sleeve 1009A, 1009B positioned external to the cannula 1000A. In alternative embodiments, the sleeves 1009 may be positioned internal to the cannula or within the walls of the cannula. In alternative embodiments, one or more spaced apart (lengthwise) collars may be employed to house the anchors 1007, rather than a sleeve. In certain exemplary embodiments, each sleeve 1009 has threads for engaging threads provided on the respective anchor to facilitate movement of the anchor relative to the sleeve and, thus, the cannula 1000A. Any number of anchors 1007 may be provided at various points about the perimeter of the distal end 1022 of the cannula 1000A. In the illustrated embodiment, for example, a first anchor 1007A is positioned to engage a first vertebra and a second anchor 1007B is positioned opposite to the first anchor 1007B to engage a second vertebra.

In alternative embodiments, the anchors 1007 may be fixed relative to the cannula 1000B. Referring to FIG. 21, for example, the distal end 1022 of the cannula 1000B may include one or more anchors 1007C, 1000D fixed to and projecting distally from the cannula 1000B.

In certain exemplary embodiments, a cannula may include a mechanism to facilitate contact between the distal end of the cannula and one or more vertebra to inhibit tissue and fluids from entering the cannula. Referring the FIG. 20, for example, the distal end 1022 of the cannula 1000A may include a gasket 1011 positioned on all or a portion of the distal end 1022 of the cannula 1000A. The gasket 1011 may be formed of a compliant material, such as a natural or synthetic rubber, silicon, bone wax or other material suitable for maintaining contact between the cannula and the vertebra.

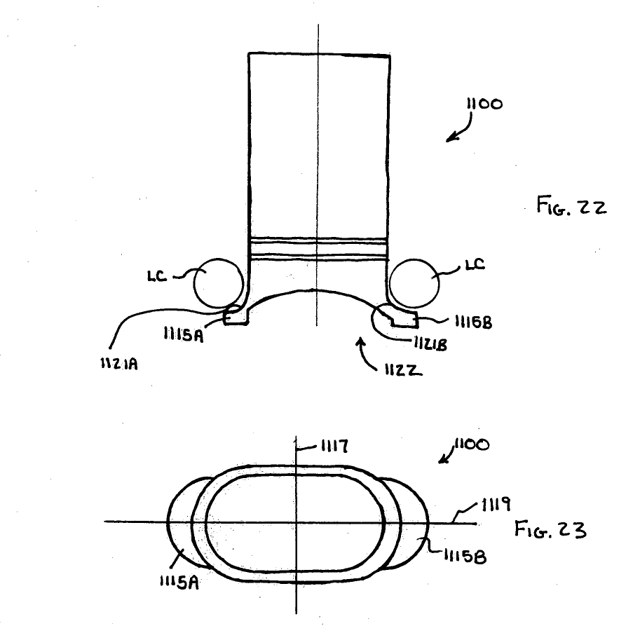

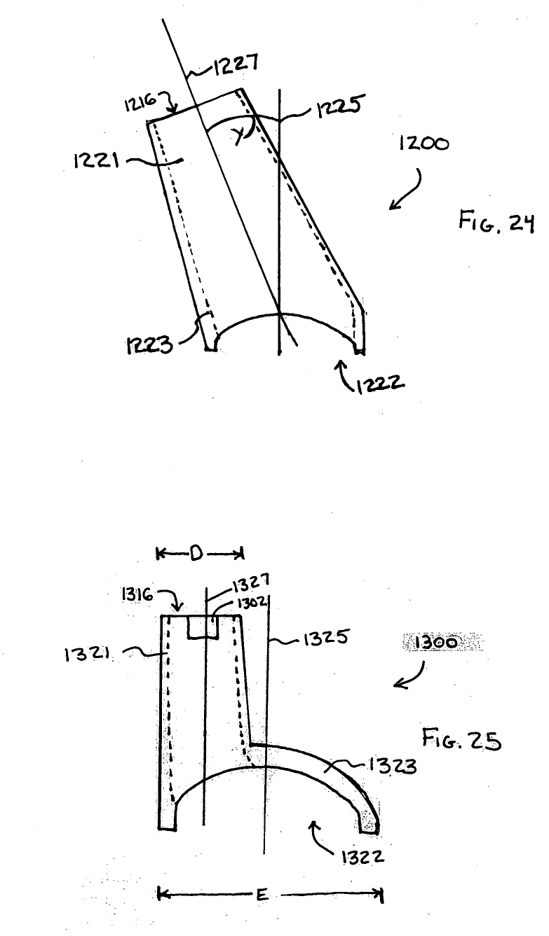

In certain exemplary embodiments, the cannula may be configured to minimize tissue damage and facilitate insertion of the cannula into the anterior cervical spine. For example, the cannula 1100 may include a pair of opposed feet 1115A,B positioned at the distal end 1122 of the cannula 1100, as illustrated in FIGS. 22 and 23. The exemplary cannula 1100 is oblong in cross section having a minor axis 117 and a major axis 1119. During insertion, the cannula 1100 may be positioned through an incision with cannula 1110 oriented such that the major axis 1119 of the cannula 1100 is oriented parallel with axis of the spine and minor axis 1117 is oriented transverse to the axis of the spine. In such an orientation, the cannula 1100 is positioned between the longus coli muscles LC. The cannula 1100 may be rotated 90°, to the position illustrated in FIGS. 22 and 23, to retract the longus coli muscles LC with the feet 1115A,B positioned beneath the longus coli muscles LC. The feet 1115A,B may have a contoured, for example, arcuate, outer surface 1121A, B to facilitate retraction of the lungs coli muscles.

In certain embodiments, the cannula may have an asymmetric construction to minimize tissue trauma while facilitating access to and maintaining contact with the selected vertebra. Referring to FIG. 24, for example, an exemplary cannula 1200 may have a proximal segment 1221 that is oriented at an angle to a distal segment 1223 of the cannula 1200. For example, the proximal segment 1221 may define a channel 1216 having a central axis 1227. In the illustrated embodiment, the distal segment 1223 includes a distal end 1222 that is contoured to correspond to the transverse curvature of the anterior surface of the vertebral body of a vertebra. The distal segment 1223 includes a central axis 1225. Upon placement of the cannula 1200 into position relative to a vertebra through an anterior approach, the central axis 1225 of the distal segment 1223 may be oriented within or generally parallel to the sagittal plane of the spine. The central axis 1227 of the proximal segment 1221 may be oriented at an angle Y to the central axis 1225 of the distal segment 1223. For a cannula designed for use in anterior approaches to the cervical spine, the angle Y may be approximately 0° to approximately 30°. Such a configuration permits the cannula 1200 to be positioned relative to the vertebra at a trajectory lateral to the midline (i.e., lateral to the sagittal plane) of the spine. In the case of the anterior cervical spine, such an off-midline trajectory may minimize trauma to the esophagus and trachea.

In alternative embodiments, the size of the proximal segment of the cannula may be reduced and the proximal segment of the cannula may be offset from the axis of the distal segment. Referring to FIG. 25, for example, the proximal segment 1321 of the exemplary cannula 1130 may be offset a distance from the central axis 1325 of the distal segment 1323. As in the exemplary cannula 1200 described above and illustrated in FIG. 24, the distal end 1322 of the distal segment 1323 of the cannula 1300 may be contoured to correspond to the transverse curvature of the anterior surface of the vertebral body of a vertebra. Upon placement of the cannula 1300 into position relative to a vertebra through an anterior approach, the central axis 1325 of the distal segment 1323 may be oriented within or generally parallel to the sagittal plane of the spine. In certain exemplary embodiments, such as the illustrated embodiment, the central axis 1327 of the channel 1316 of the proximal segment 1321 may be oriented approximately parallel to and offset from the central axis 1325 of the distal segment 1323. In alternative embodiments, the central axis 1327 of the channel 1316 of the proximal segment 1321 may be oriented at an angle to the central axis 1325 of the distal segment 1323. In the exemplary embodiment, the extent D of the proximal segment 1321 is less than the extent E of the distal segment 1323 of the cannula 1300. For a cannula designed for use in anterior approaches to the cervical spine, the extent D of the proximal segment 1321 may be approximately 5 mm to approximately 25 and the extent E of the distal segment 1323 may be approximately 15 mm to approximately 20 mm. In an anterior approach to the cervical spine, the distal segment 1321 of the cannula 1330 may be positioned beneath the esophagus and trachea, minimizing the need to retract the esophagus and trachea during the procedure. One or more proximal windows or openings 1302 may be provided to facilitate access of instruments and implants to the distal end 1322 of the cannula 1300.

What is claimed is:

1. A cannula comprising:
a proximal end,
a distal end configured to correspond to the curvature of an anterior surface of a vertebra, and
a sidewall defining a channel between the proximal end and the distal end and defining a longitudinal axis.

2. The cannula of claim 1, wherein the cannula has an elliptical cross section.

3. The cannula of claim 1, wherein the cannula has a circular cross section.

4. The cannula of claim 1, wherein at least a portion of the distal end has a shape approximate to a curvature of an anterior surface of a vertebra in the sagittal plane.

5. The cannula of claim 1, wherein at least a portion of the distal end has a shape approximate to a curvature of an anterior surface of a vertebra in the transverse plane.

6. The cannula of claim 1, further comprising a first cutout formed in the sidewall.

7. The cannula of claim 6, further comprising a second cutout formed in the sidewall, the second cutout being diametrically opposed to the first cutout.

8. The cannula of claim 7, wherein the first cutout has length along the longitudinal axis that is greater than a length of the second cutout.

9. The cannula of claim 1, further comprising surface texturing on an outer surface of the sidewall.

10. The cannula of claim 9, wherein the surface texturing comprises at least one annular groove formed on an outer surface of the sidewall.

11. A system for minimally invasive spine surgery comprising:
a first dilator having a first diameter; and
a cannula comprising
a proximal end;
a distal end spaced apart a distance from the proximal end, the distal end configured to correspond to the curvature of an anterior surface of a vertebra;
and a sidewall defining a channel extending from the proximal end to the distal end, the lumen having a length sufficient to at least span from a skin incision to proximate a vertebra and a diameter greater than the first diameter.

12. The system of claim 11, further comprising a plurality of cannulated dilators of varying lengths and diameters.

13. The system of claim 11, wherein at least a portion of the distal end has a shape approximate to a curvature of an anterior surface of a vertebra in the sagittal plane.

14. The cannula of claim 11, wherein at least a portion of the distal end has a shape approximate to a curvature of an anterior surface of a vertebra in the transverse plane.

15. A cannula comprising:
a proximal end,
a distal end, and
a sidewall defining a channel between the proximal end and the distal end, the sidewall including a distal edge that defines the distal end of the cannula, distal edge having a first segment having a shape that approximates a curvature of an anterior surface of a vertebra.

16. The cannula of claim 15, wherein the first segment has at least one arcuate section.

17. The cannula of claim 15, wherein distal edge has a second segment having a shape that approximates a curvature of an anterior surface of a vertebra.

18. The cannula of claim 15, wherein the second segment is positioned opposite the first segment and the first and second segments have an analogous shape.

19. The cannula of claim 15, wherein the distal end of the cannula includes one or more tabs for receiving a distraction pin.

20. The cannula of claim 15, wherein the sidewall of the cannula includes an opening provided therein extending from the proximal end to the distal end, the opening sized to receive a distraction pin.

21. The cannula of claim 20, wherein the opening has a cross section sized to permit motion of the distraction pin therein.

22. The cannula of claim 15, wherein the proximal end of the cannula includes a proximal opening to facilitate positioning of instruments through the cannula.

23. The cannula of claim 15, wherein the proximal end of the cannula is rotatable relative to the distal end of the cannula.

24. The cannula of claim 15, wherein the proximal end of the cannula tapers distally from a first extent to a second, lesser extent.

25. The cannula of claim 15, wherein the proximal end of the cannula includes a slot for receiving an instrument.

26. The cannula of claim 15, wherein the distal end of the cannula includes at least on anchor to facilitate connection of the cannula to one or more vertebrae.

27. The cannula of claim 26, wherein the at least one anchor is fixed to the distal end of the cannula.

28. The cannula of claim 26, wherein the at least one anchor is adjustable relative to the cannula.

29. The cannula of claim 15, wherein the distal end of the cannula includes a gasket constructed from a complaint material to facilitate connection of the cannula to a vertebra.

30. The cannula of claim 15, wherein the distal end of the cannula includes a pair of opposed feet extending from outward from the cannula.
